(19) United States
(12) Patent Application Publication
(10) Pub. No.: US 2016/0219968 A1
(43) Pub. Date: Aug. 4, 2016

(54) FOOTWEAR WITH PERFORMANCE MEASUREMENT DEVICE

(71) Applicants: Andrew Martin, Redwood City, CA (US); James Martin, Fort Collins, CO (US)

(72) Inventors: Andrew Martin, Redwood City, CA (US); James Martin, Fort Collins, CO (US)

(21) Appl. No.: 15/009,403

(22) Filed: Jan. 28, 2016

Related U.S. Application Data
(60) Provisional application No. 62/109,083, filed on Jan. 29, 2015.

(51) Int. Cl. A43B 3/00; A41B 11/00; A61B 5/00; A61B 5/11; A61B 5/103; A43B 5/14; A43B 23/26

(52) U.S. Cl. CPC A43B 3/0005; A43B 5/14; A41B 11/00; A43B 23/26; A61B 5/1118; A61B 5/1038; A61B 5/6807; A41B 2500/10; A41B 2500/20

(57) ABSTRACT

Footwear for an individual such as a cyclist is provided with a performance measurement device for measuring the individual's performance. A first exemplary article of such footwear includes a shoe in combination with an optional means for substantially immobilizing the shoe to a pedal of a cycle, e.g., a cleat, and the performance measurement device. A second exemplary article of such footwear is a sock in combination with the measurement device. Typically, the measurement device is embedded or located within the footwear and is not mounted to an exterior surface, e.g., the bottom sole surface, of the footwear.

### CROSS REFERENCE TO RELATED APPLICATIONS

This application claims priority to U.S. Provisional Application Ser. No. 62/109,083, entitled “FOOTWEAR WITH PERFORMANCE MEASUREMENT DEVICE,” filed on Jan. 29, 2015, by inventors Andrew Martin and James Martin, the disclosure of which is incorporated by reference in its entirety.

### BACKGROUND OF THE INVENTION

The present invention relates generally to an article of footwear in combination with a measurement device. More specifically, the invention relates to footwear such as a cleated cycling shoe or a sock having a performance measurement device. The device may be placed at a location other than at a cleat or at a bottom surface of the shoe. The device may be used to provide unprecedentedly accurate feedback to a cyclist so as to optimize the cyclist's overall performance during cycling activities.

An activity tracker is a wearable device that measures the activity of the tracker's wearer. For example, a FitBit® activity tracker may measure the number of steps a wearer walks, the wear's quality of sleep, and other personal metrics. As another example, U.S. Patent Application No. 20070260421 to Berner et al. describes sneakers having activity trackers included therewith.

Activity measurement may serve as an important metric for athletes. Power measurements, for example, allow for proper training, serve as a measure of general fitness and provide a vital racing metric. As a result, power meters allow for data driven decisions by athletes and coaches. For example, during endurance racing events, power meters may be used by an athlete to gauge how much effort he or she is initially putting forth so as to prevent early overexertion.

Footwear with activity trackers are known in the art. For example, U.S. Pat. No. 8,122,773 to Wyatt et al. describes a force sensing device that is placed on the bottom of the shoe. In essence, the force sensing device is mounted on the bottom surface of a shoe to measure the force exerted by the wearer of the shoe. The force sensor is pre-stressed with a compressive load to an intermediate point in its dynamic range. Strains that further compress or decompress the force sensor can thus be measured.

In a general sense, power measurement devices for cycling may be considered to be an activity tracker. Typically, power measurement devices for cycling are physically associated with a component of a cycling apparatus, e.g., a bicycle. For example, U.S. Pat. No. 8,011,242 to O'Neill et al. describes a sensor device for determining forces exerted by a cyclist on a pedal of a bicycle. The sensor device includes a plurality of sensors coupled to a substrate, and wiring coupled to the sensors and the substrate, wherein the sensors, the substrate, and the wiring are housed inside a pedal spindle coupled to the pedal.

U.S. Pat. No. 6,418,797 to Ambrosina et al. describes a power measurement device that includes a strain gauge. For example, the hub of the driven wheel of a bicycle includes one or more components such as an inner hub member, an outer hub member or a linking member which couple rotational torque through the wheel to rotate the wheel. One or more strain gauges are mounted on one of the components that couples torque through the wheel such that strain in the torque coupling component is detected. The detected strain is used to determine applied torque which is used to determine power applied to the driven wheel of the bicycle. Because the torque coupling component is mounted in the hub in the path along which torque is coupled through the wheel, the sensor rotates with the wheel as it detects the applied torque.

U.S. Pat. No. 6,356,847 to Gerlitzki describes A method for determining torque exerted on a body of revolution capable of being driven rotatably about an axis of rotation, e.g., on a bottom bracket bearing shaft of a bicycle. The method involves using measurement generators are arranged on the body of revolution at an axial distance or a radial distance from one another. Transducers are assigned to the measurement generators.

U.S. Pat. No. 6,356,848 to Cote et al. describes a method and apparatus for measuring the power output of a bicycle driven by the elongate flexible member such as chain. The method comprises measuring the speed and tension of the drive chain and calculating the power output therefrom. The apparatus includes a chain speed sensor, a chain tension sensor, and electronic processing apparatus to calculate and display the power output based upon the chain measurements.

U.S. Patent Application Publication No. 20100093494 to Smith describes an apparatus for measuring and monitoring torque exerted by a cyclist while pedaling a bicycle. The apparatus includes a cartridge or the like which is adapted to be releasably retained within a hollow spindle of the bicycle, and one or more sensor elements for progressively sensing and generating signals, during rotation of a crankshaft of the machine, which are indicative of the angular position of the or each crank arm of the bicycle and/or the torque applied thereto. The apparatus allows for measuring and monitoring both in the direction of, and against the direction of, the cyclist's pedaling.

Cycling footwear with an activity tracker is also known in the art. For example, U.S. Pat. No. 7,599,806 to Hauschildt describes a method of calculating power applied to a pedal and crank arm based drive mechanism via a force sensor located inside a shoe. The method involves receiving force signals from a force sensor inside a shoe worn by a person applying force to the pedal and crank arm based drive mechanism, using the force signals in mathematical models to calculate the power calculating an integral of a square of a derivative of the signal received from the force sensor versus time, and comparing a result to a threshold to determine whether the crank arm based drive mechanism is being rotated or is at rest. If the crank arm based drive mechanism is at rest, a cadence is set to zero, and an average of all force samples is calculated.

U.S. Pat. No. 8,762,077 to Redmond et al. describes a measurement device for measuring a cyclist's power output, in response to an external force provided by the cyclist applied to a bicycle. The device comprising a force sensor embedded in a bicycle cleat bolted to a shoe. The device also includes an accelerometer for measuring a cyclist's power output.

Nevertheless, the above-described technologies suffer from a number of drawbacks. For example, cyclists often own more than one bicycle and may not wish to use technologies that rely on sensors immobilized to only one bicycle. While such a drawback may be solved by immobilizing sensors on cycling shoes, sensors attached to cycling shoes may lack the reliability, precision, and accuracy of performance demanded by elite cyclists. For example, cleats often wear out, so sensors embedded in cleats may have to be replaced and/or calibrated often. Similarly, the bottom surfaces of shoes used in cross-country cycling are often exposed to mud, rocks, and other debris that may reduce the operational lifetime and accuracy of sensors mounted beneath the soles of the shoes.

Thus, opportunities exist for improving cycling activity trackers so as to reliably, precisely and accurately measure a cyclist's athletic performance.

### SUMMARY OF THE INVENTION

In a first embodiment, the invention provides footwear for a cyclist. Such footwear may comprise, a shoe having an interior region in which the cyclist's foot may be placed, an optional means for substantially immobilizing the shoe to a pedal of a cycle, and a measurement device associated with the shoe for measuring the cyclist's performance. Upper and sole portions, in combination, form the shoe. In some instances, the immobilizing means may include a toe clip and/or strap, optionally attached to a pedal of a bicycle. Alternatively, the immobilizing means may include a cleat for engagingly interlocking with the pedal of the bicycle, wherein the cleat immobilized to a bottom surface of the shoe. The measurement device may neither be embedded in the cleat nor mounted to the bottom surface of the footwear.

Typically, the measurement device is located within the interior region of the footwear. In addition, the sole portion may include a plurality of layers. In such a case, the measurement device is located between layers of the sole portion. Alternatively, the measurement device is embedded, removably or permanently, in the sole portion. In some instances, the measurement device is located in a wearable sock inside a shoe. Furthermore, the measurement device may be located in a tongue of the shoe.

The measurement device may include any or a combination of an accelerometer, a force sensor and a gyrometer. When a plurality of sensors is provided, the sensors may be of similar or different types.

When footwear is provided as a pair, the pair may include a left article and a right article. A pair of shoes, for example, includes at least one measurement device associated with at least one shoe for measuring the cyclist's performance. For example, left and right measurement devices may be provided for left and right shoes, respectively. The devices may independently measure the cyclist's performance for each foot, e.g., as the cyclist pull a crank upward and/or push a crank downward.

In another embodiment, a sock is provided for measuring an individual's athletic performance. The sock includes a covering for the individual's foot and a measurement device. The covering may be comprised substantially or entirely of a knitted or woven material. The covering typically extends to at least about the individual's ankle. The device is associated with, e.g., removably attached to, the covering for measuring the individual's performance.

The sock may be used by itself or be used with another wearable item, e.g., a cycling shoe. The cycling shoe may include a substantially rigid sole portion. The device may be interposed between the cyclist's foot and a sole portion of the shoe and/or between the foot and an upper portion of the shoe.

Additional embodiments, features and advantages of the invention will be apparent from the following description.

### BRIEF DESCRIPTION OF THE DRAWINGS

FIG. 1 depicts in cross-sectional view a measurement device embedded underneath the insole of a shoe.

FIG. 2 depicts in cross-sectional view a measurement device invention embedded on top of the insole of a shoe.

FIG. 3 depicts in cross-sectional view a measurement device embedded within, e.g., between an upper and a lower layer of, the insole of a shoe.

FIG. 4 depicts a sock having a measurement device associated therewith located within a shoe.

FIG. 5 depicts in cross-sectional view a measurement device embedded in a tongue of a shoe.

FIG. 6 depicts schematically the selected components of the measurement device.

FIG. 7 shows an equation of how required value relate to each other for calculating.

FIG. 8 shows how forces are applied to a shoe when a cyclist pushes a pedal downward during a downstroke portion of a crank's rotational cycle.

FIG. 9 shows how forces are applied to a shoe when a cyclist pulls a pedal upward during an upstroke portion of a crank's rotational cycle.

### DETAILED DESCRIPTION OF THE INVENTION

Definitions and Overview

Before describing the present invention in detail, it should be noted that embodiments of the invention may take the form of a useful process, machine, manufacture, or composition of matter, or any new and useful improvement thereof. It is also to be understood that the invention is not limited to specific makers of cleated footwear or particular electronic communication protocols, as such may vary. It is further to be understood that the terminology used herein is for the purpose of describing particular embodiments only, and is not intended to be limiting.

In addition, as used in this specification and the appended claims, the singular article forms “a,” “an,” and “the” include both singular and plural referents unless the context clearly dictates otherwise. Thus, for example, reference to “a shoe” includes a pair of shoes as well as a single shoe, reference to “a power measurement device” includes a single power measurement device as well as a combination of power measurement devices, and the like.

In addition, terminology indicative or suggestive of a particular spatial relationship between elements of the invention is to be construed in a relative sense rather an absolute sense unless the context of usage clearly dictates to the contrary. For example, an “upper” portion of a shoe that also includes a sole portion does not necessarily indicate that the upper portion is always located above the sole portion.

In this specification and in the claims that follow, reference is made to a number of terms that shall be defined to have the following meanings, unless the context in which they are employed clearly indicates otherwise:

The term “apparel” is used herein in its ordinary sense and generally refers to personal attire, clothing, something that adorns, and related items. For example, apparel in the form of footwear may include shoes, boots, sandals, flip flops, slippers, socks, hosiery, footed leggings, footed unitards, toe rings, ankle braces, etc.

The terms “electronic,” “electronically,” and the like are used in their ordinary sense and relate to structures, e.g., semiconductor microstructures, that provide controlled conduction of electrons, holes and/or other charge carriers.

The term “internet” is used herein in its ordinary sense and refers to an interconnected system of networks that connects computers around the world via the TCP/IP and/or other protocols. Unless the context of its usage clearly indicates otherwise, the term “web” is generally used in a synonymous manner with the term “internet.”

The prefix “micro” refers to items having dimensions on the order of micrometers or somewhat less in size or dimensions. Thus, for example, the term “microelectronic device” refers to an electronic device having features, e.g., transistors and other items of electronic circuitry, on the order of microns or submicrons. Other terms containing the prefix “micro” are to be construed in a similar manner.

The term “mobile device” is used in its ordinary sense and refers to a portable, computing device, typically less than about 1 kilogram, that is small enough to be used while held in associated with a user's foot, hand, or other body part. Typically, mobile devices are wireless in nature and are powered by one or more secondary (rechargeable) batteries, though mobile devices may be powered by primary (nonrechargeable) batteries or wired powered sources. Mobile devices of the invention may be associated with a global positioning system (GPS).

“Optional” or “optionally” means that the subsequently described circumstance may or may not occur, so that the description includes instances where the circumstance occurs and instances where it does not.

The term “shoe” is used in its ordinary sense and refers to a covering for the foot, often made of leather, with a sturdy, e.g., substantially rigid, sole. A “shoe” differs from a “boot” in that boots typically reach above the ankle. A shoe is distinguishable from a “sock” in that socks typically have no substantially rigid sole. Socks are also unlike shoes and boots in that shoes and boots typically come in left and right pairings whereas a left sock may structurally, e.g., by shape and/or constructions, be substantially identical to a right sock.

When referring to footwear described herein, the term “sole,” as in a “sole portion,” is used in its ordinary sense and refers to the lower part of footwear or the base on which an upper portion and optionally the undersurface of a foot rests. The term “sole” is not necessarily interchangeably used with the term “insole.” Typically, the term “insole” is used to refer to an item that exhibits an upper surface that lies within footwear on which the foot may rest. In contrast, the term “sole” typically refers to an item that includes a lower surface that serves as an exterior surface, e.g., tread,” of the footwear. In some instances, a sole may include an attached, detachable, and/or integrated insole layer.

The term “storage medium” is used in its ordinary sense and refers to any device or material on which data can be electronically placed, kept, and retrieved, regardless whether the data is stored permanently, e.g., via magnetic disk drives, optical disk, etc., or temporarily, e.g., by way of volatile random access memory modules.

The term “substantially rigid” is used to refer to an item whose resistance to deformation, e.g., elastic, plastic, or otherwise, is adequate for the proper functioning and/or the intended use of the invention. The term “substantial” is to be constructed in a contextual manner. For example, when accuracy is at issue, a substantially rigid sole having a measurement device physically associated therewith should have mechanical properties that allow the measurement device to generate an output that exhibits an accuracy of at least about 80%. Preferably the measurement device exhibits an accuracy of at least about 90%. Optimally, the measurement device exhibits an accuracy of at least about 95%. The terms “substantial” and “substantially” are used analogously in other contexts involving an analogous definition.

The term “wireless” is used herein in its ordinary sense and refers to any of various devices that are operated with or actuated by electromagnetic waves rather than via wire or other physical connections. Exemplary wireless protocols include, for example, ANT+ and Bluetooth.

In general, the invention relates to articles of apparel, e.g., footwear, that include one or more measurement devices. One or more component sensors in a mobile measurement device may be used to track the performance of a cyclist. A sensor, for example, may be used to determine forces and/or work generated by the cyclist while the cyclist is pedaling a bicycle. Measurements from measurement devices may be transmitted in a manner such that the cyclist and/or others receive substantially instantaneous feedback regarding the performance of the cyclist. In addition or in the alternative, data generated by a measurement device may be stored at a remote location for further analysis.

Measurement Device

The measurement device may take any of a number of forms. For example, the measurement device may be microelectronic in nature and may be comprised of a single item or a plurality of items. In some instances, the items may be modular in nature. In such a case, different modules may be detachably or permanently assembled to form a unitary item. For example, some modules may be physically associated with footwear while other modules may be physically associated with a bicycle or other gear that a cyclist may use, e.g., riding gloves, helmet, jersey, mirror, seat, frame, gears, chains, cranks, pedals, distance/speed monitor, wheel, etc. Optionally, some components of the measurement device may be microelectronic in nature while other components are not.

In any case, the measurement device may be a component of a system that includes wired and/or wireless communication means known in the art. For example, the measurement device may serve to transfer data and/or information pertaining to the user's performance to a multipurpose electronic device such as cell phone, tablet computer, or other internet-enabled or other networked apparatus. Such electronic devices typically include one or more central processing units as well as memory, e.g., in the form of storage media such as flash drives and magnetic disk and tape drives.

The measurement device may be constructed as a battery-powered mobile device. However, other energy sources may be used as well. For example, the measurement device may be powered by piezoelectric devices, solar panels and other technologies known in the art. When in use, the measurement device may be ultimately powered by the pedaling action of a cyclist or other athlete using the measurement device.

As to the workings of the measurement device, any of a number of technologies generally known in the art can be used. For example, the measurement device may operate by measuring potential and/or kinetic energy, power output, displacement, stress, strain, momentum change, etc. Such measurements may be carried out, e.g., using means that are electrical, magnetic, mechanical, chemical, thermal, acoustic, and/or optical in nature. Specific measurement devices suitable for use with the invention include, e.g., force/pressure sensors, accelerometers, and gyrometers, the general operational principles of which are described below.

A pressure sensor measures pressure, an expression of force per unit area. Numerous pressure sensing technologies are known in the art. For example, a pressure sensor may take the form of a transducer that generates a signal, e.g., an electrical signal, as a function of pressure imposed. Pressure sensors can alternatively be called pressure transducers, pressure transmitters, pressure senders, pressure indicators, piezometers, etc.

For example, pressure sensors that employ a conductive polymeric material may be used. The conductive polymer changes resistance in a predictable manner following application of force to its surface. Typically, the polymer material is formed into a sensing film that includes both electrically conducting and non-conducting particles suspended in a polymeric matrix. The particles are submicron sized, and are formulated to reduce the temperature dependence, improve mechanical properties and increase surface durability. Applying a force to the surface of the sensing film causes particles to touch the conducting electrodes, changing the resistance of the film. As with all resistive based sensors, force-sensing resistors require a relatively simple interface and can operate satisfactorily in moderately hostile environments, and may exhibit a thickness less than about 0.5 mm, low cost and good shock resistance.

An accelerometer measures acceleration. Such measurements are useful in determining changes in velocity (directly, as the acceleration is the first time derivative of the velocity) and changes in position (by integrating the signal). Accelerometers are usually used for measuring small movements. A multiple-axis accelerometer can also be used as an absolute orientation sensor in an up-down plane.

As a related matter, a gyroscope or gyrometer measures either changes in orientation (regular gyro or integrating rate gyro) or changes in rotational velocity (rate gyro).

Deployment of Measurement Device

How the measurement device of the invention is deployed represents an important aspect of the invention. For example, force and/or pressure sensors may be used determine the force applied by the cyclist's foot. The force sensor may be placed beneath the foot to determine the force applied during the down pedal stroke. A force sensor may be placed in the tongue of the shoe to determine how much force is generated during the up pedal stroke. The down pedal force sensor may be placed beneath the sole of the shoe, embedded in the sole of the shoe or above the sole. The total force can be calculated individually per foot and per system which comprises of both feet.

Another important aspect of the invention relates to how the performance measurement device relates to footwear associated therewith. For example, when a pressure sensor is used, it is important that the sensor be placed at a location of the footwear that allows the sensor to provide meaningful data. In some instances, an effectively rigid sole surface may be required to ensure proper operation of the sensor. However, a rigid surface may compromise the comfort level of the athlete wearing the footwear. Thus, in some instances, the measurement device may be interposed between a rigid surface and a compliant surface, the rigid surface ensuring the proper operation of the sensor, the compliant surface providing a degree of cushioning for the athlete's foot.

In some instances, calibration of the inventive device may be required. Calibration parameters may include, for example, cyclist weight, bicycle weight, crank length, wheel diameter, etc. Such calibration may be needed to effect algorithmic calculations to determine metrics such as cyclists uphill, downhill, forwards or backwards speed. In some instances, the algorithm may be comprised of data from accelerometers in each of a pair of left and right shoes. Because each accelerometer gathers data in x, y and z planes, tandem data sets can determine how fast and grade a cyclist is going uphill, downhill, forwards or backwards. As bicycle pedals are typically offset by 180 degrees, revolutions per minute or cadence can be determine based on the position of each pedal. Cadence is also an important measurement when riding, training or racing.

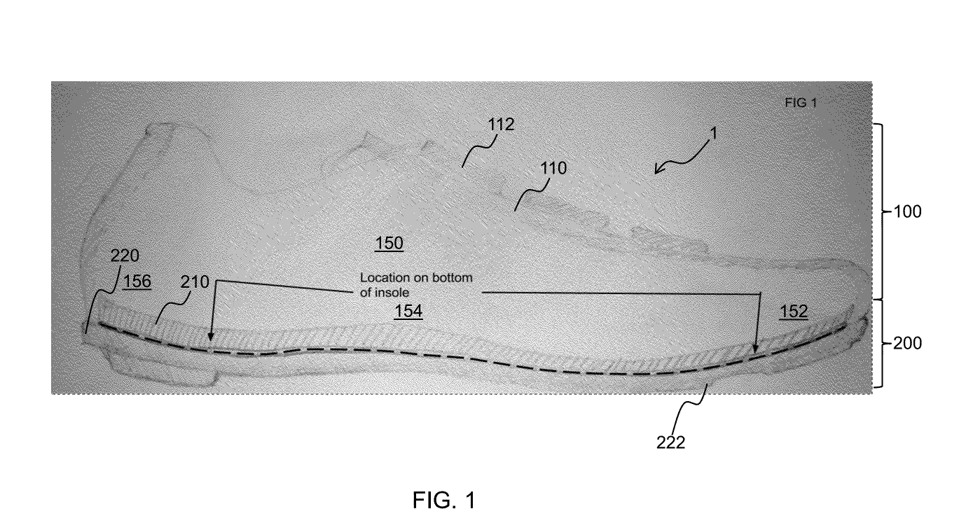

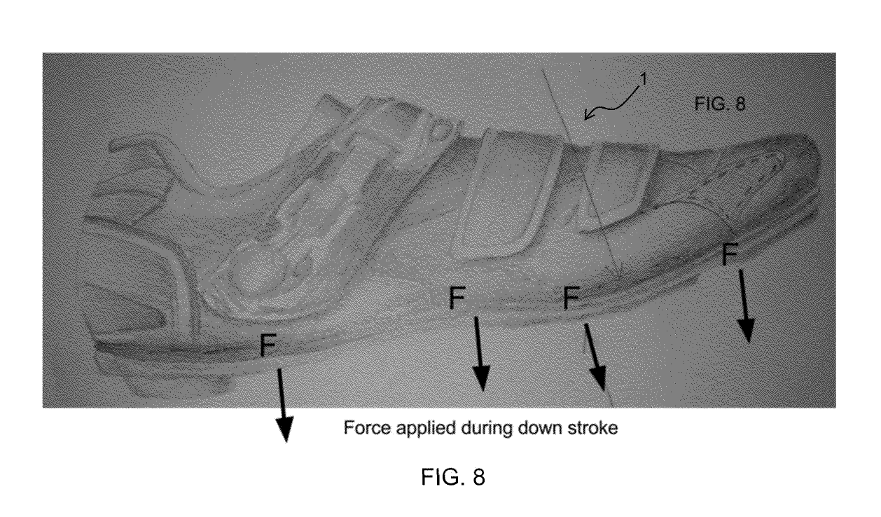

FIGS. 1-9 depict various aspects of the invention. As with all drawings referenced herein, in which like parts are referenced by like numerals, it should be noted that the drawings are not necessarily to scale and certain features of the invention may be exaggerated or omitted for clarity of presentation.

FIG. 1 depicts in cross-sectional view an article of the inventive foot apparel in the form a cycling shoe 1 comprising an upper portion 100 and a lower (sole) portion 200. The upper portion 100 includes a tongue 110 and an optional closure 112, e.g., a shoe lace or hook-and-loop fastener marketed under the trademark Velcro®. A sole portion 200 lies below and is attached to the upper portion 100 such that the upper portion's periphery bounds substantially or entirely the insole 210. A lower surface of the insole 210 is attached to an upper surface of the shoe's sole 220. Together, the upper and lower portions of the shoe form an interior shoe region 150 into which a foot may be inserted. The interior shoe region 150 may be generally divided into a toe region 152 at the front of the shoe, a heel region 156 at the back of the shoe, and an arch region 154 in between the front and back of the shoe.

Optionally, an immobilizing means (not shown) may provided with the inventive shoe. For example, a cleat may be positioned beneath the ball of the shoe wearer's foot. Cleats are typically attached to the lower (exterior) surface 222 of the sole 220.

As indicated by the dashed line in FIG. 1, a measurement device or a component thereof may be embedded underneath the insole of a shoe. That is, a measurement device may be positioned between the insole and the sole of the shoe beneath the interior shoe region 150. Depending on the nature of the measurement device, the precise location of the measurement device may vary. In some embodiments, the measurement device may be placed directly beneath a ball region of the shoe wearer's foot.

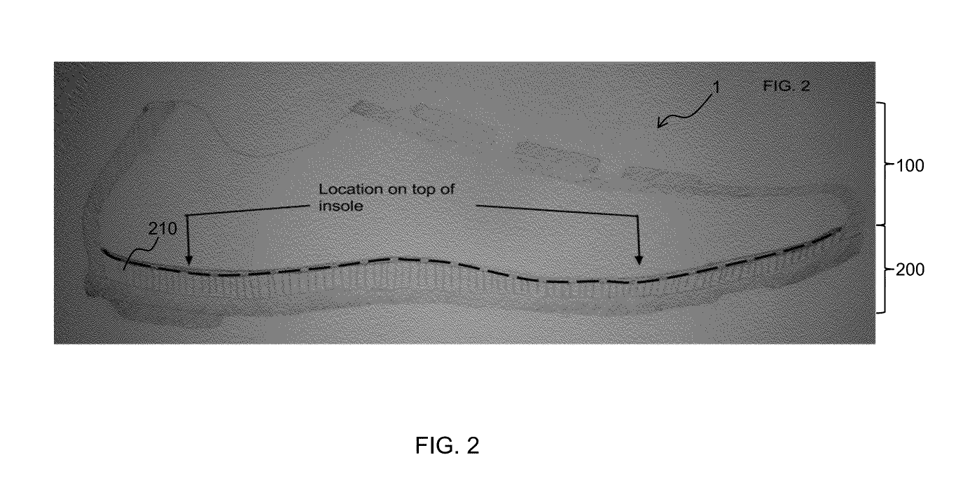

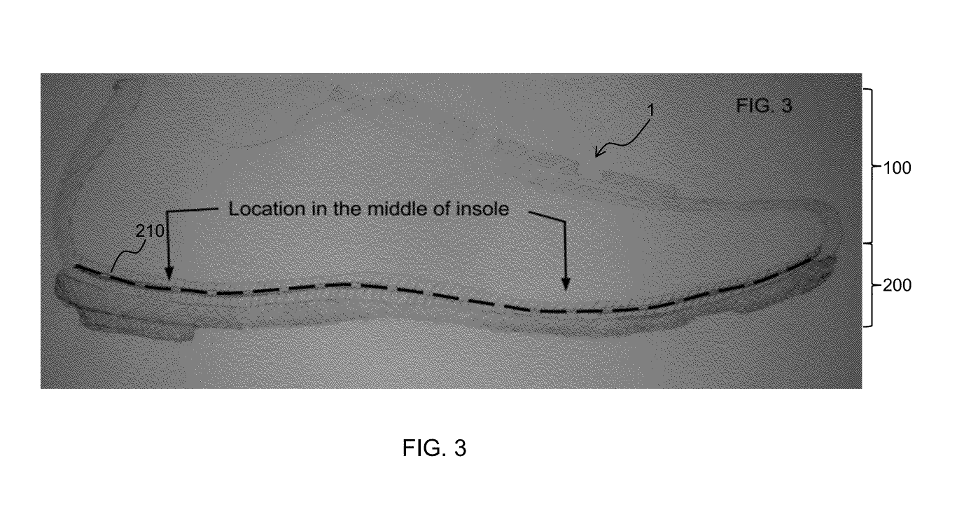

FIGS. 2 and 3 depict alternative embodiments of the invention similar to that depicted in FIG. 1 in that the measurement device thereof is physically associated with at least the insole of the shoe. As shown by dashed lines in FIG. 2, the measurement device or a component thereof may be located at or near the top surface of the shoe's insole. In addition or in the alternative, e.g., as shown in FIG. 3, the measurement device may be embedded within the insole of the shoe. In such a case, the insole may include an upper and lower layer of the same or different materials.

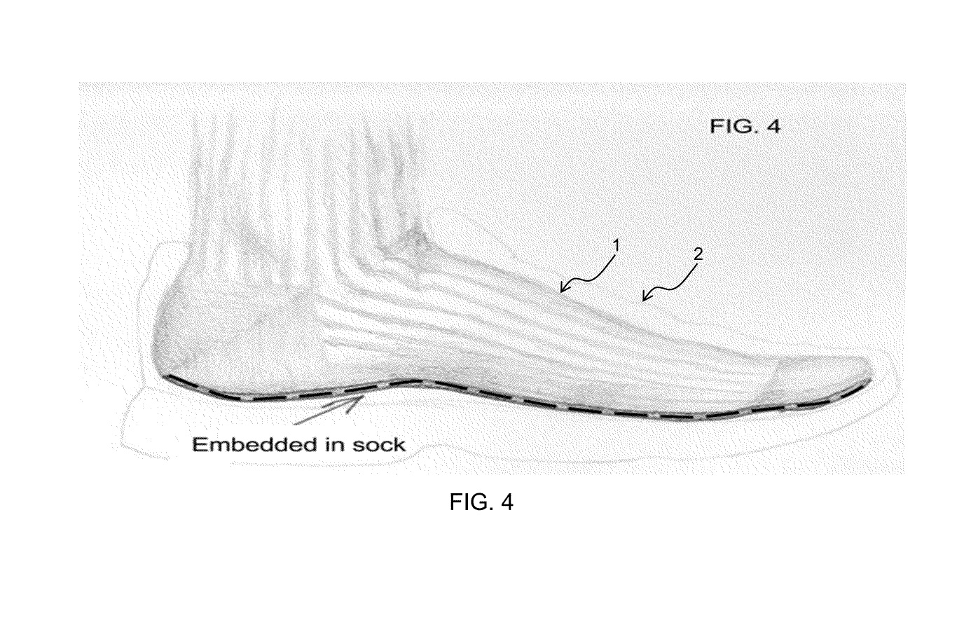

The invention is not limited to shoes. FIG. 4 shows an embodiment of the invention in the form of a sock 1 having a measurement device (as indicated by the dashed line) associated therewith at a lower portion thereof. Typically, the sock is used in conjunction with a shoe 2. In such a case, the shoe may exhibit mechanical properties, e.g., sufficient rigidity or elasticity, to facilitate the operation of the measurement device. In addition, the sock may be washable. Regardless whether the invention takes the form of a shoe sock, and/or other type of apparel, the measurement device may be water proof and/or reside in a pocket that allows for the device to be moved prior to washing.

In any case, FIGS. 1-4 and 8 show similar embodiment of the invention in which a measurement device may be placed under a cyclist's foot during cycling. For example, as the cyclist pushes a pedal downward, a force sensor is located beneath the cyclist's foot of a cyclist may be used, the sensor can measure the force that is applied by a cyclist's foot when pedaling down.

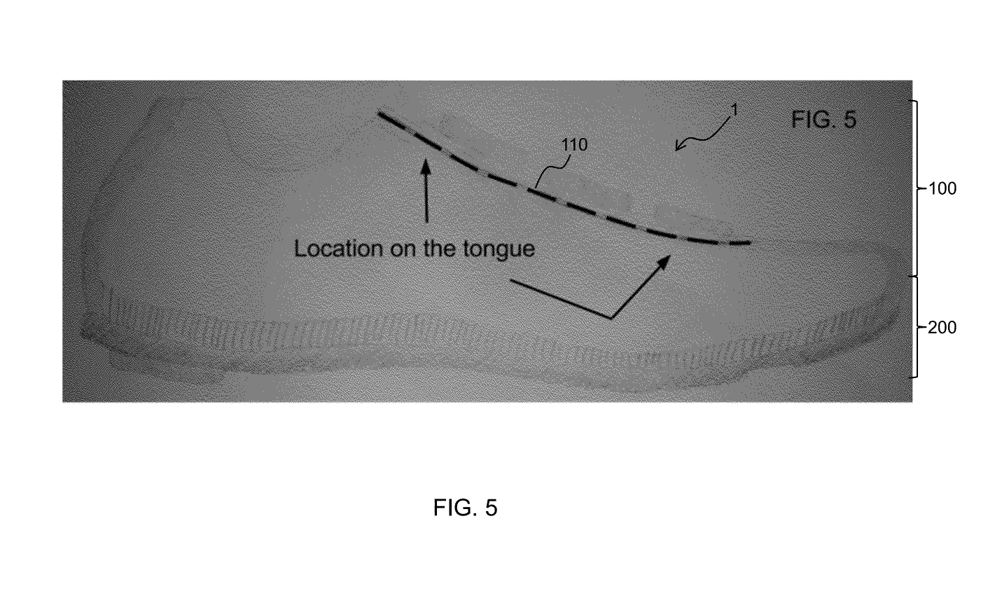

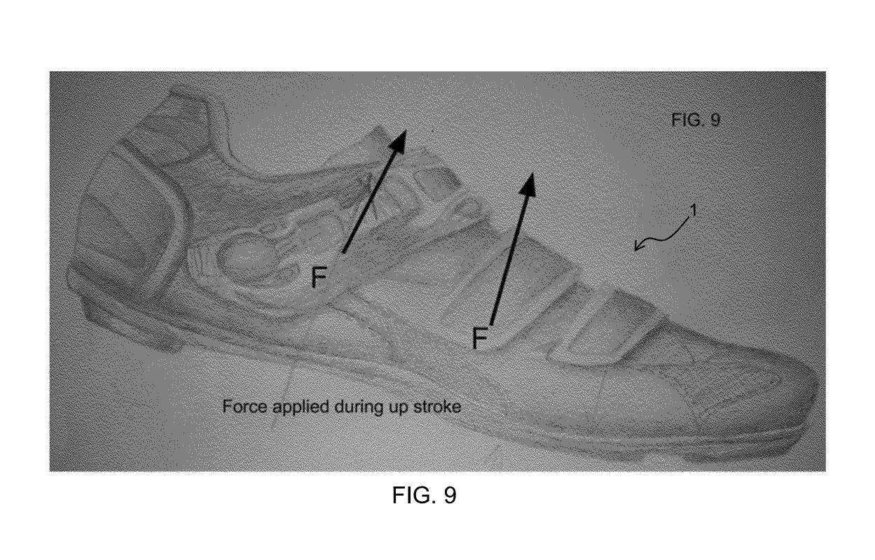

In contrast, FIGS. 5 and 9 show an embodiment invention that may be used when forces are applied as a cyclist pulls a pedal upward during an upstroke of a crank's rotational cycle. As depicted in FIG. 5 a measurement device may be embedded in a tongue 110 of a shoe 1. Physical association of a measurement device with the tongue of a shoe may allow a cyclist to calculate and monitor how much force is applied during the upstroke.

As discussed above, the invention may include one or more measurement devices. Thus, the invention may include one or more measurement device located below an athlete's foot, as shown in FIGS. 1-4, and 8, as well as a measurement device located above the athlete's foot, as shown in FIGS. 5 and 9. In addition or in the alternative, sensors and/or components thereof may be physically associated with the inventive footwear at a location corresponding to the sides of the athlete's foot.

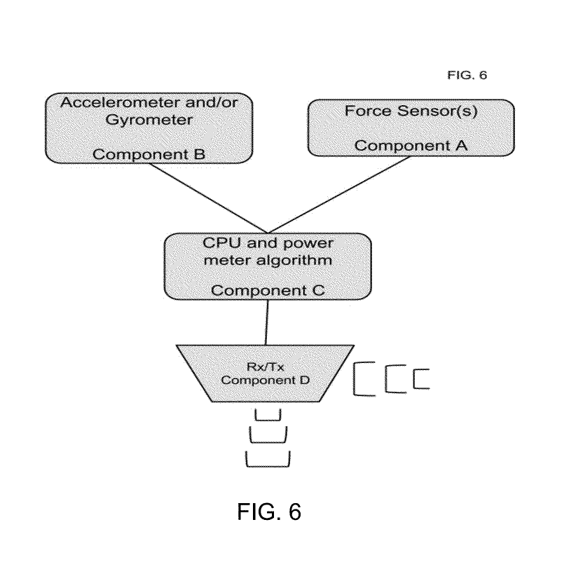

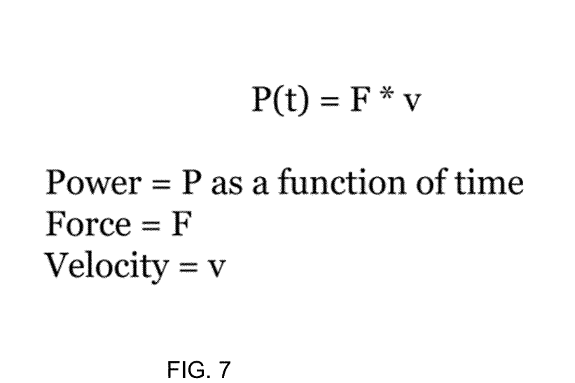

FIG. 6 depicts schematically the selected components of an exemplary measurement device that may be used in conjunction with cycling. As shown, the device may include, Component A—force sensor(s), (2) Component B—accelerometer and/or gyrometer, Component C—central processing unit (CPU) adapted to use input from Components A and B and a power meter algorithm, e.g., one that involves the equations depicted in FIG. 7. Component D—wireless transmitter/receiver (Tx/Rx), may be used to provide input and output to the CPU. Component D may include subcomponents such as antenna, ANT+ and/or Bluetooth technology.

Any of a number of algorithms may be used to calculate metrics useful for assessing athletic performance. For bicycling, power (FIG. 7) is a key metric for assessing performance. Other important performance metrics, related or not to power, include distances, speed, time pedaled, etc. Algorithms for calculating such metrics can be derived from known laws of physics in view of the implementation of the invention as discussed above.

Other measures of athletic performance include pulse rate, blood pressure, breathing rate, etc., e.g., as correlated with the metrics discussed above. Such measures may be obtained using measurement devices near the foot of a user of the invention or elsewhere, e.g., at the wrist, arm, chest, etc. Such measurement devices may be constructed as other articles of apparel as well, e.g., in the form of a bracelet, glove, shirt, pants, etc.

Thus, the invention provides a number of advantages over known technologies. For example, the invention allows elite cyclists to fine tune their athletic performance so as to provide maximum athletic efficiency. When measurement devices are used in both left and right articles of footwear, power balance measurements may be made for interpretation by athletes, coaches and/or medical professionals. In addition, the invention allows cyclists to use the same article of footwear for multiple bicycles. Rather than requiring specialized algorithms or expensive replaceable bicycle components, the simplicity of the invention provides economic and performance advantages over known measurement devices.

Variations on the invention will be apparent to persons of ordinary skill in the art. For example, the measurement device may exhibit any of a number of form factors. In some instances, the measurement device itself may exhibit a certain degree of flexibility itself, even if certain components of the device may be substantially rigid. In addition or in the alternative, the measurement device may be sufficiently small so as to represent an unobtrusive item that does not inhibit the operation of a bicycle or the performance of a cyclist.

In addition, the invention is not limited to measurement devices that are associated in their entireties with a shoe or a sock. For example, the measurement device of the invention may include a pressure sensor and a velocity sensor. The pressure sensor may be placed, e.g., on the insole of the shoe. An accelerometer or velocity sensor may be placed on the crank of a bicycle rather than be associated directly with foot and/or shoe.

Notably, embodiments of the invention in the form of a shoe typically do not depart from the basic anatomy of a shoe. For example, all shoes have a sole. When various layers are used, soles may include an insole, optional midsole, and an outsole, wherein the insole (also known as a sock liner) forms the interior bottom of a shoe, the outsole is the layer that may be placed in direct contact with the ground, and the midsole is interposed between the outsole and the insole.

While the invention has been illustrated and described in detail in the drawings and foregoing description, the same is to be considered as illustrative and not restrictive in character, it being understood that only the preferred embodiment has been shown and described and that all changes and modifications that come within the spirit of the invention are desired to be protected. The invention may include or exclude certain elements, e.g., as discussed above. For example, while the invention has generally been described in terms of shoes and socks, the invention is not limited to articles of apparel that cover or leave bare a cyclist's ankle. As another example, not all embodiments of the invention are limited to footwear for cyclists. Similarly, while certain aspects of the invention have been actually reduced to practice, portions of the invention have been described in theoretical terms. Neither the theoretical portions of the disclosure nor the portions of the disclosure actually reduced to practice contained herein are meant to be limiting.

All patents and publications referenced herein are incorporated by reference to the fullest extent practicable not inconsistent with the above disclosure.

What is claimed is:

1. An article of footwear for a cyclist, comprising,
an upper portion;
a sole portion, in combination with the upper portion, forming a shoe having an interior region in which the cyclist's foot may be placed;
an optional means for substantially immobilizing the shoe to a pedal of a cycle; and
a measurement device associated with the shoe for measuring the cyclist's performance, wherein the measurement device is neither embedded in the immobilizing means nor mounted to a bottom exterior surface of the footwear.

2. The article of claim 1, comprising the immobilizing means.

3. The article of claim 2, wherein the immobilizing means comprises a cleat for engagingly interlocking with the pedals of the cycle, the cleat immobilized to the bottom surface of the shoe.

4. The article of claim 2, wherein the immobilizing means comprises a top clip and/or strap substantially immobilized to the pedals of the cycle.

5. The article of claim 1, wherein the measurement device is effective to generate data used to determine metrics selected from cycle cadence, uphill speed, downhill speed, forward speed and backward speed

6. The article of claim 1, wherein the measurement device is located within the interior region of the footwear.

7. The article of claim 1, wherein the sole portion includes a plurality of layers and the measurement device is located between layers of the sole portion.

8. The article of claim 1, wherein the measurement device is embedded in the sole portion.

9. The article of claim 1, wherein the measurement device is located in a wearable sock inside the shoe.

10. The article of claim 1, wherein the upper portion includes a tongue and the measurement device is located in the tongue.

11. The article of claim 1, wherein the measurement device comprises an accelerometer.

12. The article of claim 11, wherein the measurement device further comprises a force sensor.

13. The article of claim 11, wherein the measurement device further comprises a gyrometer.

14. The article of claim 1, wherein the measurement device comprises a plurality of sensors.

15. The article of claim 14, wherein the sensors are selected from an accelerometer, a force sensor, and a gyrometer.

16. The article of claim 14, wherein the sensors are of different types.

17. Articles of footwear for a cyclist, comprising,
left and right upper portions;
left and right sole portions, in combination with the left and right upper portions, respectively, forming left and right shoes having interior regions in which the cyclist's feet may be placed;
optional cleats for engagingly interlocking with pedals of a cycle, the cleats immobilized to bottom exterior surfaces of the shoes; and
at least one measurement device associated with a shoe for measuring the cyclist's performance, wherein neither measurement device is embedded in any cleat nor mounted to the bottom exterior surface of any shoe.

18. The articles of claim 17, comprising left and right measurement devices associated with the left and right shoes, respectively, for measuring the cyclist's performance.

19. The articles of claim 17, wherein the left and right measurement devices independently measure the cyclist's performance for each foot.

20. A sock located within a shoe for measuring an individual's athletic performance, comprising:
a covering for the individual's foot comprised substantially or entirely of a knitted or woven material, the covering extending to at least about the individual's ankle; and
a measurement device associated with the covering for measuring the individual's performance,

21. The sock of claim 20, wherein the shoe is a cycling shoe.

22. The sock of claim 21, wherein the cycling shoe includes a substantially rigid sole portion.

23. The sock of claim 20, wherein the measurement device is interposed between the foot and a sole portion of the shoe.

24. The sock of claim 20, wherein the measurement device is interposed between the foot and an upper portion of the shoe.
